(19) United States
(12) Patent Application Publication
(10) Pub. No.: US 2003/0118673 A1
(43) Pub. Date: Jun. 26, 2003

(54) Smilagenin and anzurogenin-D for the treatment of alzheimer's disease

(76) Inventors: Zongqin Xia, Shanghai (CN); Yaer Hu, Shanghai (CN); Ian Rubin, Nottingham (GB); Jonathan Brostoff, London (GB); Brian Whittle, East Yorkshire (GB); Weijun Wang, Huntingdon (GB); Phil Gunning, Cambridge (GB)

Correspondence Address: OPPENHEIMER WOLFF & DONNELLY LLP, Attn: Spyros J. Lazaris, Suite 3800, 2029 Century Park East, Los Angeles, CA, 90067, US

(21) Appl. No.: 10/336,176

(22) Filed: Jan. 3, 2003

Related U.S. Application Data
(63) Continuation of application No. 09/647,128, filed on Jan. 11, 2001, now abandoned.
Application No. 09/647,128 is a 371 of international application No. PCT/GB99/00960, filed on Mar. 26, 1999.

(30) Foreign Application Priority Data
Mar. 26, 1998 (GB) 9806513.9
Mar. 8, 1999 (GB) 9905275.5

(51) Int. Cl. A61K 35/78; A61K 31/58

(52) U.S. Cl. 424/753; 514/173

(57) ABSTRACT

The invention discloses the use of smilagenin and anzurogenin D in the treatment of cognitive dysfunction and similar conditions. Methods of treatment, and pharmaceutical compositions are also disclosed.

### BACKGROUND OF THE INVENTION

The present invention relates to smilagenin and anzurogenin-D and their use in treating cognitive disfunction and allied conditions; and to compositions for use in such treatments. The invention is also concerned with the treatment of conditions that are characterised by a deficiency in the number or function of membrane-bound receptors.

### SUMMARY OF THE INVENTION

In the following, the present invention will be described principally with reference to the treatment of Alzheimer's disease (AD) and senile dementia of the Alzheimer's type (SDAT), where deficiencies in a number of receptor types have been demonstrated. However, it is to be understood that the present invention relates generally to the treatment of conditions attributable to intrinsic pathological conditions and/or exposure to adverse environmental conditions these conditions being characterized by a deficiency in the number or function of membrane-bound receptors or a deficiency in transmission at the junctions between neurons or at the junctions of neurons and effector cells.

Conditions of the type mentioned above include Parkinson's disease, Lewi body dementia, postural hypertension, autism, chronic fatigue syndrome, Myasthenia Gravis, Lambert Eaton disease, diseases and problems associated with Gulf War Syndrome, occupational exposure to organophosphorus compounds and problems associated with aging.

Alzheimer's disease (AD) and senile dementia of the Alzheimer's type (SDAT) are grave and growing problems in all societies where, because of an increase in life expectancy and control of adventitious disease, the demographic profile is increasingly extending towards a more aged population. Agents which can treat, or help in the management of, AD/SDAT are urgently required.

Age-associated memory impairment (AAMI) is a characteristic of older patients who, while being psychologically and physically normal, complain of memory loss. It is a poorly defined syndrome, but agents which are effective in treatment of AD/SDAT may also be of value in these patients.

Research into AD/SDAT is being carried out by traditional and conventional medical research methods and disciplines. In conventional medicine, there are several approaches to the treatment of AD/SDAT. It is known that the biochemical processes subserving memory in the cerebral cortex are (at least in part) cholinergically-mediated. Those skilled in the art will know that “cholinergically mediated” mechanisms may be directly attributable to acetylcholine acting on receptors, and these are direct effects. Other, clinically useful effects may also be caused by modulation of release of acetylcholine from pre-synaptic nerve endings or inhibition of enzymes that destroy acetylcholine. These modulating factors may be exerted through neurons where the mediator is non-cholinergic; these are referred to as indirect effects. Some attempts at treatment have focussed on the role of other mediators such as 5-hydroxytryptamine, which is a mediator in other areas of brain, such as the mid-brain nuclei. However, since fibers from these areas are projected forward into the cerebral cortex where the primary transmitter is acetylcholine, attention has focussed on the management of this mediator in the search for appropriate therapeutic agents.

Cholinergic strategies for the treatment of AD/SDAT have been directed at several points along the pathway of formation, synaptic release and removal of released acetylcholine.

One approach involves treatment with high doses of lecithin and other precursors of acetylcholine. This is of limited use in producing sustained improvements in cognitive performance.

Another approach involves the use of vegetable drugs such as Polygalae root extract, which has been shown to enhance choline-acetylcholine transferase (CAT) activity and nerve growth factor (NGF) secretion in brain. Oral administration of NGF has no effect on central nervous system neurons because it is a high molecular weight protein that cannot pass through the blood-brain barrier. However, agents which can pass through the blood-brain barrier and have a stimulating effect on NGF synthesis in the central nervous system have been proposed for the improvement of memory-related behavior.

The results of a third clinical approach, which uses cholinesterase inhibitors such as tacrine hydrochloride, have been marginally more positive than the above. Substances obtained from plants used in Chinese and Western medicine, for example huperzine, galanthamine, and physostigmine have all been shown to be of some—although limited—benefit in the treatment of AD/SDAT in clinical studies and also in laboratory models. All of these substances are inhibitors of acetylcholine esterase (ACNE). In patients with AD/SDAT, there may be reduced synthesis of acetylcholine (ACh), reduced efficiency in release of ACh from presynaptic stores, and a decrease in the number or function of postsynaptic (M) receptors. Reductions in pre-synaptic MZ receptors have also been shown. The beneficial effect of AChE inhibitors is attributed to enhancement of acetylcholine levels at synapses in brain by slowing down the destruction of released transmitter.

### DETAILED DESCRIPTION OF PREFERRED EMBODIMENTS

Compositions which modulate cholinergic function are known to affect memory and recall. For example, nicotine stimulates nicotinic acetylcholine receptors, and the short lived memory enhancing effects of cigarette smoking are thought to be due to the effect of nicotine. Scopolamine, an antagonist of acetylcholine, will produce amnesia and impaired cognitive function manifesting in psychomotor tests as a prolongation of simple reaction times, possibly as a result of impaired attention, and is used for this purpose as an adjunctive analgesic treatment. The amnesic effect of scopolamine can be antagonized by nicotine.

There are two families of nicotinic receptor subtypes (a and 0), and each includes four subgroups which differ in ligand specificity. The role of nicotinic receptors in the CNS is not well understood at the molecular level. It is possible that agents binding to nicotinic receptors may modify the rate of turnover at muscarinic receptor sites in brain. Nicotinic receptors are ligand-gated ion channels, and their activation causes a rapid (millisecond) increase in cellular permeability to Na′ and Ca″, depolarization and excitation.

Another class of cholinergic receptors can be stimulated by muscarine. Such muscarinic (M) receptors are G protein-coupled receptors. Responses of muscarinic receptors are slower; they may be excitatory or inhibitory. They are not necessarily linked to changes in ion permeability. Five types of muscarinic receptors have been detected by cholinergic receptor cloning, and are designated as ml-ms. Pharmacological effects are associated with four of the cloned receptors and they are designated as M,-M4 based on pharmacological specificity.

Using specific receptor proteins and monoclonal antibodies, it has been possible to further localize muscarinic receptors in brain as m, (postsynaptic) and m2 (presynaptic). In heart, M2 receptors are postsynaptic. Presynaptic muscarinic receptors are thought to be inhibitory, the binding of ACh to these receptors attenuating the release of further ACh to provide a negative feedback mechanism for ACh release. Selective M2 receptor antagonists which are preferentially distributed to the brain may therefore be useful in treating Alzheimer's disease.

It is known that, in disease states such as AD/SDAT, there is general neuronal loss and deficits in cholinergic nerve function. It has been speculated that the high affinity nicotinic binding sites in the remaining cholinergic neurons might be converted to low affinity binding sites in treating such diseases, thereby sustaining transmitter release. By lowering the affinity of the nicotinic binding sites, a quick desensitizing process is avoided.

Agonist activation at nicotinic receptors in brain has rapid onset and offset. A decreased affinity of the nicotinic receptors will reduce the desensitization process. Schwarz R. D. et al (J. Neuro Chem 42, (1984), 1495-8) have shown that nicotine binding sites are presynaptically located on cholinergic (and also 5-hydroxytryptaminergic and catecholaminergic) axon terminals. A change in high affinity binding sites on AD/SDAT may also induce a change in the modulatory effect the nicotinic binding sites may have on other transmitter systems.

Presynaptic cholinergic mechanisms are also under inhibitory control by GABAergic neurons and this inhibition is thought to be intensified in AD/SDAT. Removal or reduction of this inhibition intensifies presynaptic cortical cholinergic activity and enhances cognitive processing.

The interactions of interneuronal fibers innervated by nicotine (reducing binding affinity), and dis-inhibition of GABAergic fibers both have a presynaptic locus.

This is a simplistic model. of central. transmission, but provides a framework for understanding the attempts which have been made to increase the effective concentration of acetylcholine in central synapses. This further illustrates the concept of direct and indirect action. There are disadvantages attaching to the three conventional therapeutic approaches to AD/SDAT treatment mentioned above: ACh precursor supplementation, agonist replacement and acetylcholine esterase inhibition. These treatments may result in a short-term increase in the availability of ACh which may activate feedback mechanisms resulting in the desensitization of postsynaptic receptors. On theoretical grounds, long term benefits would not be predicted and when treatment is interrupted, any benefits in management of AD/SDAT and AAMI disappear and the condition may even be aggravated.

It has been shown that a compound with M, agonist and M2/M3 antagonist activity improved cognitive performance in SDAT patients (Sramak et al, Life Sciences vol. 2, No. 3, 195-202, 1997). However, this compound causes unacceptable cholinergic side effects, such as fatigue, diarrhea and nausea.

A more radical approach to AD/SDAT and AAMI aims to increase the number of postsynaptic (M) receptors, in brain. It is known from Chinese Patent No. CN1096031A, that sarsasapogenin (SaG) can up-regulate M, cholinergic receptors and also down-regulate (i.e. move towards normal levels of) P-adrenergic receptors, the number of which may be pathologically-raised in AD/SDAT.

Patent applications have been published which claim the usefulness of a number of steroid sapogenins having spirostane, furo-spirostane, spirosolane or solanidine structures in the treatment of diseases including SDAT. Two patent publications are of particular relevance here: Chinese patent application No. CN1096031A discloses two-way regulatory effects of spirostane sapogenin, sarsasapogenin, on β-adrenergic and M-cholinergic receptors. The disclosure in this document, however, is brief. The other document of relevance is patent publication DE 4303214A1 which claims the use of a very wide range of saponins and sapogenins in the treatment of a whole range of diseases that the inventors consider to be of viral origin. This disclosure is however of dubious value in that it is well recognized that there is no infective element to a very large number of the conditions that are characterised by deficient synaptic transmission and thus the basic premise of the alleged invention is flawed. In addition they present no data of any kind that allows one skilled in the art to be able select a preferred compound from the large number that are claimed.

The inventors have found that smilagenin (SMI) and anzurogenin D (AZD) exhibit the ability to regulate receptors. In particular, these compounds—and especially SMI—have been found to increase the number of M2 receptors in the brain. Thus, according to one aspect of the invention, there is provided the use of smilagenin and/or anzurogenin D in the manufacture of a medicament for the treatment of a condition characterised by a deficiency in postsynaptic membrane-bound receptor number or function.

Those skilled in the art will be aware of the relationship between saponins and their sapogenins, and that the desired effects of sapogenins can be exhibited in patients by administration of the corresponding saponins, or a mixture thereof. Hydrolysis of at least a proportion of saponin occurs in the gastrointestinal tract. The skilled man will also be aware of the epimerisation of certain sapogenins under conditions of acid hydrolysis.

The sapogenins of interest in this invention are of the following general formula:

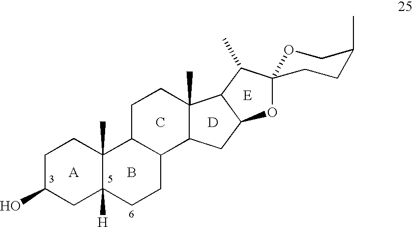

With reference to this general formula, SMI and AZD have the structure indicated in the Table below:

| | A/B ring | C25 methyl | |
|---|---|---|---|
| | Cis/Trans/ | stereochemistry | Hydroxyl group(s) |
| Compound | unsaturation | (R or S) | on Spirostane Ring |
| Smilagenin | Cis | R | 3β-OH |
| Anzurogenin-D | Trans | R | 3β-OH, 5α-OH |
| | | | 6β-OH |

The saponins and sapogenins of interest in the present invention occur naturally in a range of plant species, notably from the genera Smilax, Asparagus, Anemarrhena, Yucca and Agave. The species presently of greatest interest include Smilax regelii Kilip & Morton—commonly known as Honduran sarsaparilla; Smilax aristolochiaefolia Miller—commonly known as Mexican sarsaparilla; Smilax ornata Hooker—commonly known as Jamaican sarsaparilla; Smilax aspera—commonly known as Spanish sarsaparilla; Smilax glabra Roxburgh; Smilax febrifuga—Kunth—commonly known as Ecuadorian or Peruvian sarsaparilla; Anemarrhena asphodeloides Bunge; Yucca schidigera Roezl ex Ortgies; and Yucca brevifolia Engelm.

According to a further aspect of the present invention, there is provided a pharmaceutical composition having cognitive function enhancing properties which comprises an effective amount of smilagenin and/or anzurogenin D.

In another aspect, the invention provides a pharmaceutical composition having cognitive function enhancing properties which comprises an effective amount of smilagenin and/or anzurogenin D in the form of an extract derived from a plant of the genus Smilax, Asparagus, Anemarrhena, Yucca or Agave.

It will be appreciated that the invention embraces within its scope the use of the compositions defined above. Thus, according to a fifth aspect, the present invention provides a method of enhancing cognitive function which comprises administering to a human or animal an effective dosage of a composition of the invention.

The invention also provides a method of enhancing cognitive function in a human or non-human animal, which comprises administering an effective dose of smilagenin and/or anzurogenin D. The smilagenin and/or anzurogenin D may, for example, be administered in a food product or beverage.

As used herein, the term “cognitive function” refers to functions such as thinking, reasoning, remembering, imagining and learning.

In identifying compounds that would have use in the treatment of SDAT and other diseases characterised by reductions in receptor numbers or synaptic transmission, the inventors have given consideration to the need to identify compounds that would have the desired effect but would be devoid of any estrogenic effects, as these would be unacceptable, particularly in male patients. A number of the compounds claimed to have activity in patent application DE 4303214A1 have marked estrogenic activity and are therefore unacceptable. Smilagenin and anzurigenin D, however, do not display estrogenic activity. In addition these two compounds were tested at other steroid receptors and neither compound was found to have any activity at any of the following receptors:

Progesterone

Glucocorticoid

Testosterone

The selected compounds have also been tested for their activity in a number of in-vitro assays. The assays/experiments that were considered of key importance in determining possible activity in the elevation of membrane bound receptor numbers were as follows:

1. Chinese hamster ovary (CHO) cells transfected with the a DNA fragment coding for a muscarinic receptor. The cell line used for the majority of the experiments was a cell line expressing the m2 receptor.

2. The effects of muscarinic receptor expression in cultured cell lines of neuronal origin were investigated.

3. Cultured cardiac muscle cells obtained from neonatal Sprague Dawley rats. The cardiac muscle cells express muscarinic receptors, typically m2. The level of these receptors falls on prolonged culture and the effects of compounds of interest in preventing the fall in receptor numbers was investigated.

The methods and the results of these experiments are now described in turn.

1 CHO cell line experiments The effects of various compounds on the expression of m2 receptors on CHO cells transfected with DNA for the m2 receptor were investigated. Receptor. numbers were assayed using tritiated QNB binding and subtracting non-specific binding. Compounds were dissolved in DMSO and DMSO was used as a control. Compounds were tested at a range of final concentrations. Compounds were also tested in the presence and absence of tamoxifen to try to distinguish an estrogen receptor mediated mechanism. The results are summarized in the Table 2 below, where the compounds used in the invention appear in bold, and data on other sapogenins is given for comparative purposes:

TABLE 2
Effects of compounds on the expression of m, receptors on CEO cells

| | | Effect on receptor |
|---|---|---|
| | Molar | expression—given as % |
| | concentration | increase compared to control |
| Compound | of compound | (negative values in brackets) |
| Sarsasapogenin | 10⁻⁵ | 34 |
| | 10⁻⁶ | (14) |
| Anzurogenin D | 10⁻⁵ | 22 |
| | 10⁻⁶ | (26) |
| Sisalgenin | 10⁻⁵ | NS |
| | 10⁻⁶ | NS |
| Smilagenin | 10⁻⁵ | 57 |
| | 10⁻⁶ | 18 |
| Diosgenin | 10⁻⁵ | NS |
| | 10⁻⁶ | NS |
| Ruscogenin | 10⁻⁵ | (22) |
| | 10⁻⁶ | NS |
| Tigogenin | 10⁻⁵ | NS |
| | 10⁻⁶ | NS |

NS = No significant effect

Thus the experiments indicate that smilagenin and anzurogenin D were able to increase the number of muscarinic receptors expressed on the surface of CHO cells cultured in-vitro. The effect was not antagonized by tamoxifen, indicating that the mechanism involved did not involve the estrogen receptor.

3 Effects of compounds on cultured cardiac muscle cells. Cardiac muscle cells were isolated from the ventricular muscle of neonatal Sprague Dawley rats using standard techniques. Cells were cultured in vitro and muscarinic receptor numbers expressed on cell surfaces membrane fragments after homogenization of cells harvested at various time points were estimated using specific binding of tritiated QNB. Preliminary experiments demonstrated that the number of receptors expressed tended to decline after 10 days of culture. The experiments were therefore designed to investigate the effects of the various compounds in inhibiting this decline in receptor numbers.

The results of these experiments are summarized in Table 3, where the compounds used in the invention appear in bold, and data on other sapogenins is given for comparative purposes:

TABLE 3
Effects of various compounds on muscarinic receptor
expression on cultured cardiac muscle cells

| | Concentration of |
|---|---|
| | compound causing a significant increase in |
| | in number of receptors expressed on neonatal |
| Compound | cardiac muscle after 10 days in vitro culture |
| Diosgenin | NS |
| Anzurogenin D | 10⁻⁶ M |
| Ruscogenin | NS |
| Sarsasapogenin | 10⁻⁵ M |
| Tigogenin | NS |
| Astragaloside | 10⁻⁵ M |
| Smilagenin | 10⁻⁶ M |

NS = No significant effect

operate through an effect on G protein and that the effects on receptor numbers are secondary to an effect on G-protein. When a membrane bound G-protein linked receptor is stimulated two basic sets of events are initiated: the effecter response; and the internalization of the receptor. The subsequent processing of the receptor to the state where it is again in a form on the cell surface or other membrane surface where it can interact with another receptor ligand appears to be subject to a number of factors. A number of these factors or mechanisms appear to be G-protein linked. There is evidence that activation of m3 receptors may have an effect on G-protein expression or levels. It is speculated that the actions of the compounds described in this patent may due to an interaction in the processes of receptor regeneration, G-protein linkage or G-protein homeostasis.

An alternative hypothesis is that the compounds are increasing the synthesis or release or a decreased rate of degradation of neurotropic factors such as brain derived growth factor and/or nerve growth factor. These effects on growth factors might be due to an effect of the compound on a cytosolic or nuclear receptor or the binding of a compound to a promoter region with a consequent effect directly on the rate of production of mRNA for the growth factor or as a consequence of increasing the production of another material factor such as G-protein or finally the effects may be secondary to an effect on receptor or G-protein procession.

1. A method for increasing the muscarinic receptor number or enhancing the function of muscarinic receptors in a human or non-human animal, comprising the administration of an effective amount of smilagenin, or anzurogenin-D or a combination thereof.

2. The method according to claim 1, which is for treating cognitive disfunction.

3. The method according to claim 1, which is for enhancing cognitive function in a patient suffering from age-related cognitive disfunction.

4. The method of claim 1 which is for treating a disease chosen from: Alzheimer's disease, senile dementia of the Alzheimer's type, Parkinson's disease, Lewi body dementia, postural hypertension, autism, chronic fatigue syndrome, Myasthenia Gravis, Lambert Eaton disease, diseases and problems associated with Gulf war syndrome, occupational exposure to organophosphorus compounds and problems associated with aging.

5. The method of claim 1 which is for treating a disease chosen from Alzheimer's disease and senile dementia of the Alzheimer's type.

6. The method of claim 1, wherein smilagenin and/or anzurogenin-D is in the form of an extract derived from a plant of the genus Smilax, Asparagus, Anemarrhena, Yucca or Agave.

7. A pharmaceutical composition which comprises a pharmacologically effective amount of anzurogenin D.

8. A pharmaceutical composition according to claim 7, which further comprises a pharmacologically effective amount of smilagenin.

9. A method of enhancing cognitive function in a human or non-human animal, which comprises administering an effective dose of smilagenin and/or anzurogenin D.

10. The method of claim 9, wherein smilagenin and/or anzurogenin-D is administered in a food product or beverage.

11. The use of smilagenin and/or anzurogenin-D in a food product or beverage to enhance cognitive function.

12. A method of enhancing cognitive function in a human or non-human animal, which comprises administering to the human or non-human animal an effective dose of a compound selected from the group consisting of smilagenin and anzurogenin-D.

13. A method as claimed in claim 12, wherein the compound is in the form of an extract from a plant of the genus Smilax, Asparagus, Anemarrhena, Yucca or Agave.

14. A method as claimed in claim 12, wherein the animal is a human suffering from Alzheimer's disease or SDAT.

15. A method as claimed in claim 12, wherein the animal is a human suffering from Parkinson's disease.

16. A method as claimed in claim 12, wherein the animal is a human suffering from Lewi Body dementia.

17. A method as claimed in claim 12, wherein the animal is a human suffering from postural hypertension.

18. A method as claimed in claim 12, wherein the animal is a human suffering from autism.

19. A method as claimed in claim 12, wherein the animal is human suffering from chronic fatigue syndrome.

20. A method as claimed in claim 12, wherein the animal is a human suffering Myasthenia Gravis.

21. A method as claimed in claim 12, wherein the animal is a human suffering from Lambert Eaton disease.

22. A method as claimed in claim 12, wherein the animal is a human suffering from Gulf War Syndrome.

23. A method as claimed in claim 12, wherein the animal is a human suffering from occupational exposure to organophosphorus compounds.

24. A method as claimed in claim 12, wherein the animal is a human in old age.

25. A method as claimed in claim 12, which comprises administering a foodstuff or beverage containing an effective dosage of a compound selected from the group consisting of smilagenin and anzurogenin-D.

26. A method as claimed in claim 12, which comprises administering a pharmaceutical composition containing an effective dosage of a compound selected from the group consisting of smilagenin and anzurogenin-D.

27. A method as claimed in claim 12, wherein the compound is anzurogenin-D.

28. A method as claimed in claim 27, wherein the anzurogenin-D is present with smilagenin.

29. A method as claimed in claim 25, wherein the compound is anzurogenin-D.

30. A method as claimed in claim 29, wherein the anzurogenin-D is present with smilagenin.

31. A method as claimed in claim 26, wherein the compound is anzurogenin-D.

32. A method as claimed in claim 31, wherein the anzurogenin-D is present with smilagenin.
